(19) United States
(12) Patent Application Publication
(10) Pub. No.: US 2016/0270865 A1
(43) Pub. Date: Sep. 22, 2016

(54) REUSABLE CATHETER WITH DISPOSABLE BALLOON ATTACHMENT AND TAPERED TIP

(71) Applicant: Auris Surgical Robotics, Inc., San Carlos, CA (US)

(72) Inventors: Casey LANDEY, San Francisco, CA (US); Jeffery B. ALVAREZ, Redwood City, CA (US); Stephen Charles DAVIES, El Dorado Hills, CA (US)

(21) Appl. No.: 15/071,908

(22) Filed: Mar. 16, 2016

Related U.S. Application Data
(60) Provisional application No. 62/134,350, filed on Mar. 17, 2015.

(51) Int. Cl. A61B 34/30; A61B 1/005; A61B 1/04

(52) U.S. Cl. CPC A61B 34/30; A61B 1/04; A61B 1/0053; A61B 2034/301; A61B 2017/00323

(57) ABSTRACT

Endoscopic surgical apparatuses and methods are described. An endoscopic apparatus comprises an elongate shaft, an inflatable element, an instrument base, and an imaging source. The elongate shaft comprises a steerable distal tip, locating feature(s) proximal of the steerable distal tip, inflation inlet(s) adjacent the locating feature(s), and a working channel. The inflatable element is slid over the elongate shaft to removably couple to the elongate shaft at the locating feature(s), thereby placing the inflatable element in fluid communication with the inflation inlet(s). The instrument base is coupled to a proximal end of the elongate shaft and can be coupled to a robotic system. The robotic system articulates the steerable distal tip through the instrument base to perform a procedure in an anatomical lumen. The robotic system can also advance and retract the endoscopic apparatus through the anatomical lumen. The imaging source is located at the steerable distal tip.

### CROSS-REFERENCE TO RELATED APPLICATIONS

This application claims the benefit of U.S. Provisional Application No. 62/134,350, filed Mar. 17, 2015, which application is incorporated herein by reference.

The present invention relates to endoscopic instruments, tools, and methods that may be incorporated into a robotic system, such as those disclosed in U.S. patent application Ser. No. 14/523,760, filed Oct. 24, 2014, U.S. Provisional Patent Application No. 62/019,816, filed Jul. 1, 2014, U.S. Provisional Patent Application No. 62/037,520, filed Aug. 14, 2014, and U.S. Provisional Patent Application No. 62/057,936, filed Sep. 30, 2014, the entire contents of which are incorporated herein by reference.

### BACKGROUND OF THE INVENTION

1. Field of the Invention

The field of the present invention relates to flexible endoscopic tools that may be used in a number of endolumenal procedures. More particularly, the field of the invention pertains to flexible endoscopic tools that incorporate disposable balloon attachments as a means of isolating the region near the distal tip.

2. Description of the Background Art

Endoscopic surgery has precipitated the development of novel technologies. In the context of bronchoscopy, there is a growing interest in using endoscopic tools to treat potentially cancerous lesions and tumors within the lungs. Current technologies, however, provide limited vision capabilities, and practitioners are often left guessing their location within the patient's lungs and aiming at a guesstimate of the lesion. Current technologies are even further limited with respect to bronchioles on the periphery, where the small size of the bronchioli is beyond the resolution of current CT and optical imaging techniques. This presents a serious problem; numerous tumors and lesions develop on the periphery of the lungs and require early diagnosis and treatment in order to prevent the spread of any cancerous cells.

Additionally, navigation with current endoscopic technologies leave much to be desired. Today's endoscopic devices are typically handheld devices with numerous levers, dials, and buttons for various functionalities, but offer limited articulation. In order to control the endoscope, physicians must manipulate levers and/or dials in concert with twisting the shaft of the scope. These techniques require the physician to contort their hands and arms while using the device in order to deliver the scope to the desired position. The resulting arm motions and positions are awkward for physicians; maintaining those positions can also be physically taxing. A robotically controlled solution would dramatically improve ergonomics and usability for the physicians.

Accordingly, there is a need for a robotic endoscopic tool that is capable of providing real-time video feedback of the interior of the bronchioles, especially in the periphery of a patient's lung.

### BRIEF SUMMARY OF THE INVENTION

In general, the present invention provides an endoscopic tool that that incorporates a balloon applicator attachment that enhances video capture and procedural efficiency. In one aspect, the present invention provides for a medical instrument that comprises an elongated, flexible shaft, and an inflation inlet along the length of the shaft, configured to convey fluid in order to inflate an elastic object. In one aspect, the elastic object is a balloon. In one aspect, the present invention further comprises an instrument base that is configured to interface with a robotic system. In one aspect, the shaft is configured to be articulated in response to rotation motion transmitted from the robotic system to the instrument base.

In yet another aspect, the present invention further comprises locating features that are configured to hold the elastic object around the inflation inlet when the object is inflated. In one aspect, the elastic object comprises of openings that are configured to be held in place by a pair of locating features on the shaft. In one aspect, the elastic object comprises an applicator that is configured to provide a lower friction surface for loading the object on the shaft. In one aspect, the applicator is permeable to water.

In yet another aspect, the present invention provides for a method that comprises inserting an elongated medical instrument into an anatomical lumen, wherein the elongated instrument comprises of a distal tip and an elastic object surrounding an inflation inlet located on the length of the instrument; positioning the distal tip at an operative site; inflating the object by conveying fluid through the inflation inlet, such that the inflated object blocks the anatomical lumen; irrigating the operative site; performing an operative procedure at the operative site; and deflating the object. In one aspect, the irrigation of the operative site is performed by an irrigation lumen that is located at the distal tip of the elongated instrument.

### BRIEF DESCRIPTION OF THE DRAWINGS

The invention will be described, by way of example, and with reference to the accompanying diagrammatic drawings, in which:

FIG. 1A illustrates a robotically-driven endoscopic instrument that incorporates an inflation inlet, in accordance with an embodiment of the present invention;

FIG. 1B illustrates the distal end 104 of the endoscopic instrument 101 from FIG. 1A, in accordance with an embodiment of the present invention;

FIG. 1C illustrates the endoscopic instrument 101 with a disposable balloon disposed around the inflation inlet 109, in accordance with an embodiment of the present invention;

FIG. 1D illustrates the endoscopic instrument 101 from FIGS. 1A, 1B with uninflated balloon 107 disposed around the inflation inlet 108, in accordance with an embodiment of the present invention;

FIG. 1E illustrates the endoscopic instrument 101 from FIGS. 1A, 1B, 1D with inflated balloon 111 disposed around the inflation inlet 108, in accordance with an embodiment of the present invention;

FIG. 2 illustrates a protocol for using the endoscopic device with a balloon attachment, such as endoscopic device 101 and balloon 111 from FIG. 1A-1E, within an anatomical lumen, to prevent unwanted fluid in non-operative regions in accordance with an embodiment of the invention; and

FIG. 3 illustrates use of an endoscopic device with a balloon attachment within an anatomical lumen.

### DETAILED DESCRIPTION OF THE INVENTION

Although certain preferred embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

In clinical applications, the use of a distally-located camera at the tip of the endoscope often provides significant visual feedback to the user, allowing the physician to successfully navigate, operate, and treat pre-determined operative regions within a patient. In the context of certain clinical procedures, however, vision may be impeded for a number of reasons, including the presence of mucus. In bronchoscopy, for example, vision may be obscured when mucus clings to the lens of the distally-located camera, obscuring large portions of the lung from the physician. Additionally, mucus may also obscure light sources, greatly reducing the visibility within the bronchioli.

Among other reasons, vision may be improved by filling the anatomical lumen (a peripheral bronchiole in the context of bronchoscopy) with fluid, such as water or saline, which distends small airways, prevents debris from obscuring the camera, and improves optical performance. Unchecked use of fluid, however, is however undesirable. For example, in bronchoscopy, when flooding bronchioles in the lung's periphery during a clinical procedure, fluid in the lung periphery often flows out of the area of interest, requiring that a constant stream of fluid to keep the area flooded. As a result, the patient's lungs often get irrigated with more fluid than initially intended for the operative region. Relatedly, filling a patient's lungs with too much fluid can be dangerous.

Accordingly, the present invention provides an efficient, disposable design for an endoscopic tool that isolates the irrigation of a target operative region.

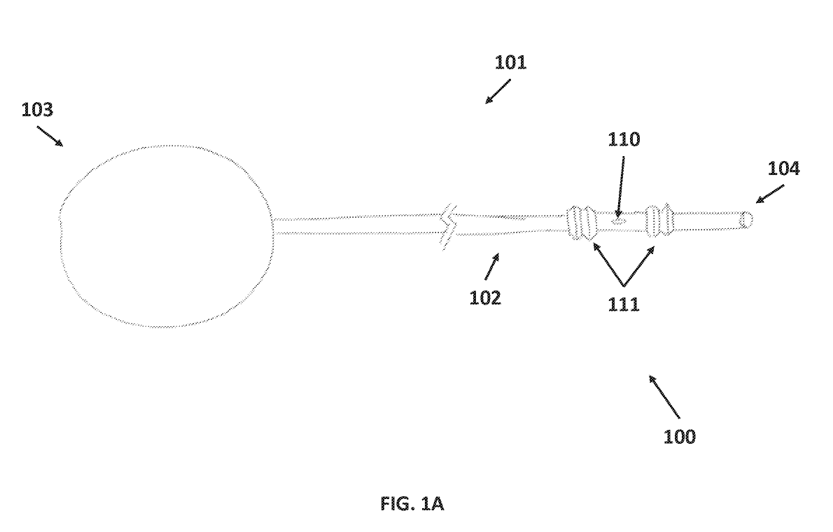

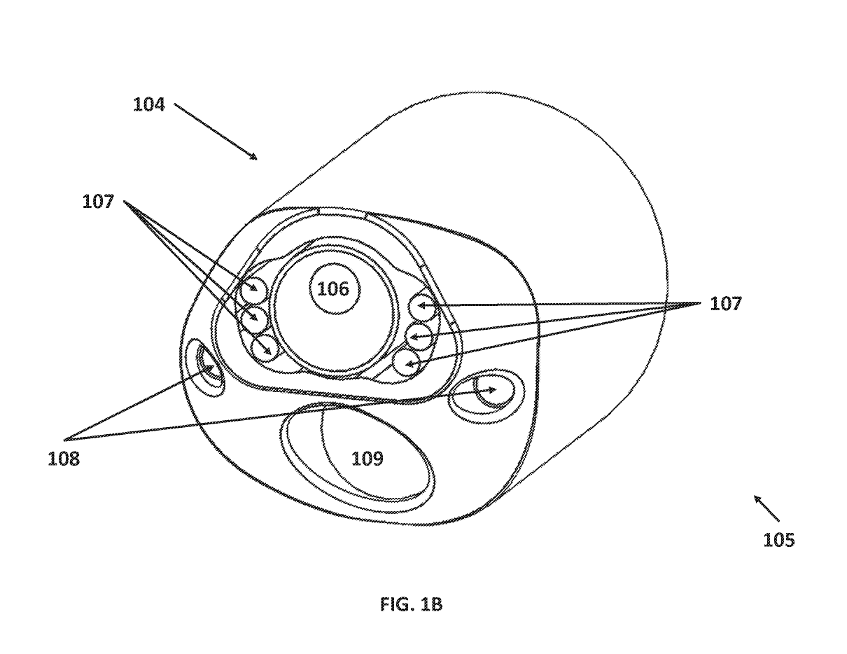

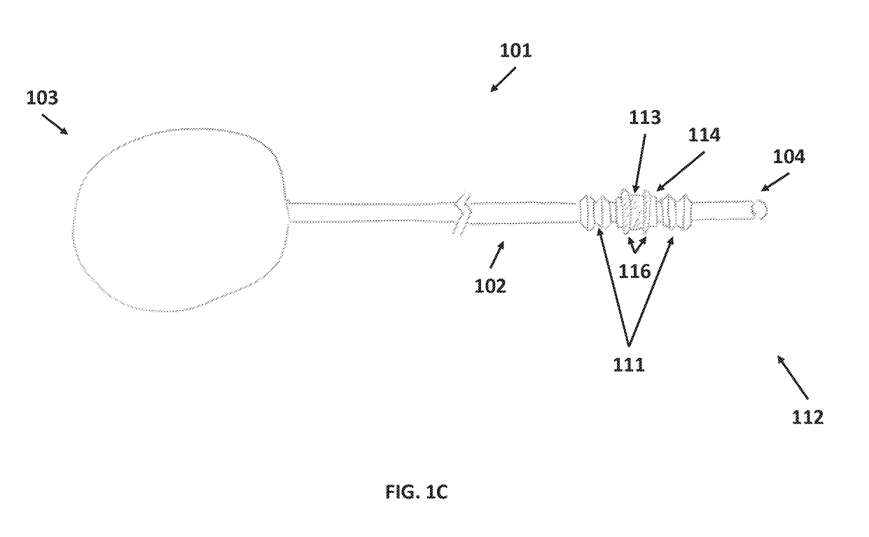

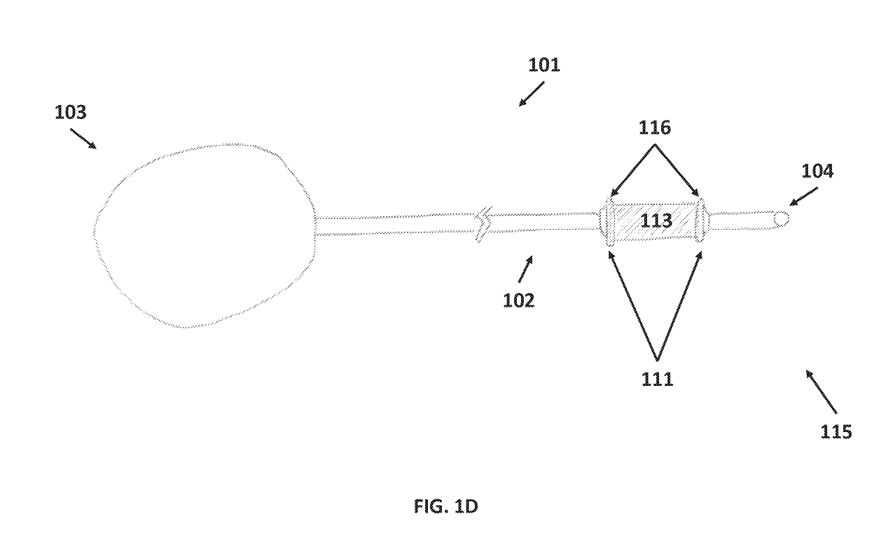

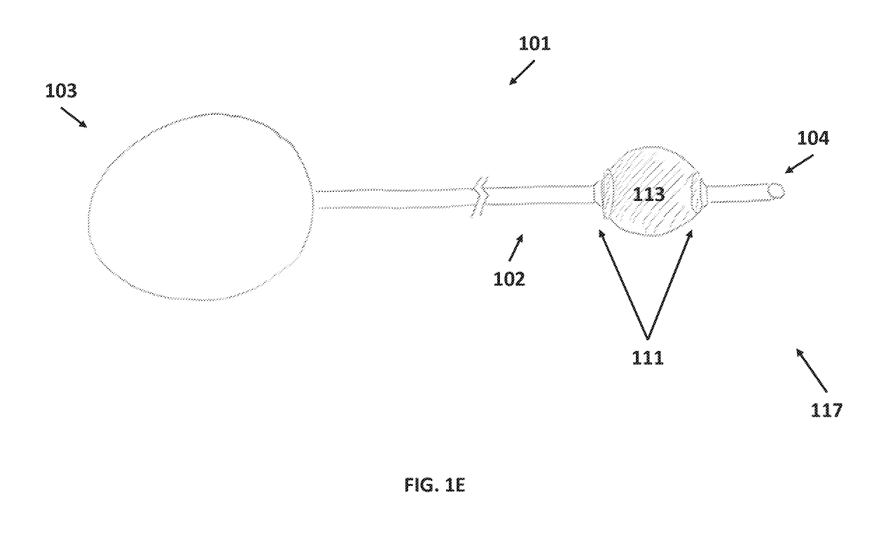

FIG. 1A illustrates a robotically-driven endoscopic instrument that incorporates an inflation inlet, in accordance with an embodiment of the present invention. As shown in view 100 from FIG. 1, endoscopic instrument 101 principally incorporates a flexible, elongated shaft 102 and an instrument base 103. In some embodiments, the endoscopic instrument 101 as a whole is reposable, i.e., usable for several procedures after sterilization. The endoscopic instrument 101 may be configured to be used within a larger robotic system, such as those disclosed in the aforementioned patent applications. In some embodiments, the instrument base 103 of the endoscopic instrument 101 may be configured to interface to the robotic instrument drive mechanism through a sterilizable interface that may incorporate a disposable drape.

The elongated shaft 102, or “catheter”, may generally be constructed by any of the manufacturing techniques disclosed in the aforementioned patent applications. Like the embodiments discussed in the aforementioned patent applications, pull wires may be run the length of the elongated shaft 102 and be fixedly coupled to the distal end such that tension on those pull wires results in articulation of the elongated shaft 102.

FIG. 1B illustrates the components located at the distal tip 104 of elongated shaft 102, in accordance with an embodiment of the present invention. As shown in view 105 from FIG. 1B, the distal tip 104 of elongated shaft 102 may comprise a visual sensing means, such as a digital camera 106. The distally-located camera 106 may be helpful for visual feedback and to assist the physician with navigation within the patient's anatomical lumens. The camera 106 may necessitate the incorporation of wires down the length of the elongated shaft 102 to convey visual data from the distal end 104 to the instrument base 103 and ultimately to the robotics platform that drives the instrument base 103. Accompanying the camera 106 at the distal tip 104 may be one or more light emitting means, such as light-emitting diodes 107, that are configured to assist the camera with visualizing the interior of the anatomical lumens. Additionally, there may be one or more channels that may be used to accommodate irrigation from the distal tip 104, such as irrigation ports 108. In this embodiment, aspiration of the irrigated fluid may be managed through aspiration from the working channel 109. Working channel 109 may also be configured to be used with tools, end effectors, and other payloads.

In some embodiments, the ports 108 may lead to combination aspiration/irrigation channels. However, the dual-purpose aspiration/irrigation channels may suffer from latency issues in comparison to dedicated aspiration channels and dedicated irrigation channels. For example, when changing functionality from irrigation to aspiration, the entire channel may need to aspirated prior to aspirating any external fluid. Similarly, when changing from aspiration to irrigation, fluid will only irrigate from the port after first flooding the entire channel first.

Among other reasons, the tapered shape of the distal tip 104, where the camera protrudes from the tip 104 as it tapers downwards towards the working channel 109, improves cannulation within anatomical lumens. Additionally, the reduced surface area around the lens of camera 106 reduces the amount of undesirable debris that may cling to the camera 106 and thus obscure its vision. Polishing the tip or applying a surface finish may be employed to further enhance these properties. The distal tip 104 is also designed to reach small anatomical spaces-the components at the distal tip 104 may be manufactured to a 3.3 mm outside diameter or less using 3/16″ steel material. The distal tip 104 may also be composed of other materials used in catheter construction, such as polyether ether ketone (PEEK).

Returning to FIG. 1A, the elongated shaft 102 may also comprise an inflation inlet 110 and a pair of balloon locating features 111. The inflation inlet 110 may be various shapes and sizes that may be appropriate for conveying fluid out of the elongated shaft 102. The inflation inlet may be fed by an inflation lumen (now shown) that may be embedded within the length of the elongated shaft 102. The inflation inlet 110 may be configured to convey fluid from the robotic system. In some embodiments, the inflation inlet 110 may run to the instrument base 103, where a fluid access port in the base 103 may interface with the robotically-provided fluid source. In some embodiments, the fluid source may be external to the robotic system. In some embodiments, the fluid source may be manually-driven and controlled.

FIG. 1C illustrates the endoscopic instrument 101 with a disposable balloon disposed around the inflation inlet 110, in accordance with an embodiment of the present invention. As shown in view 112, a disposable balloon 113 may be slidingly disposed over the distal end of the endoscopic instrument 101 and positioned over the inflation inlet 110. Balloon 113 may have two ends, each with an elastic opening 114 to provide a tight fit over the elongated shaft 102. The balloon 113 may be loaded onto the endoscopic device 101 by sliding the balloon 113 over the elongated shaft 102 from the distal tip 104. To facilitate loading, balloon 113 may further comprise an interior applicator tube 114 that may be positioned over the inflation inlet 110. The applicator tube 114 may provide a lower friction surface for easier loading of the balloon 113 onto the elongated shaft 102. Applicator tube 114 may also be fluid permeable to allow fluid from the inflation inlet 110 to fill the balloon 113.

FIG. 1D illustrates the endoscopic instrument 101 from FIGS. 1A-1C with uninflated balloon 113 disposed around the inflation inlet 110, in accordance with an embodiment of the present invention. As shown in view 115, after positioning the balloon 113 over the inflation inlet 110, the ends of the balloon 113 may be unwrapped or stretched such that the elastic openings 116 of the balloon couple with the balloon locating features 111 that flank the inflation inlet 110. The balloon locating features 111 are configured to anchor the ends of (disposable) balloon 113 that may be wrapped around the inflation inlet 110. When positioning the balloon 113, the balloon locating features 111 may be helpful to ensure that the balloon 113 is correctly aligned over the inflation inlet 110. The balloon locating features 111 are configured to tightly couple with the elastic openings 116 of the balloon 113 such that they maintain their grip and hold the balloon 113 in place even under pressure and/or if the balloon 113 is inflated. The balloon locating features may be in a variety of forms configured to anchor the ends of the disposable balloon 113, including ridges or indentations, which may be cornered, sharp, or smooth.

FIG. 1E illustrates the endoscopic instrument 101 from FIGS. 1A-1D with inflated balloon 113 disposed around the inflation inlet 110, in accordance with an embodiment of the present invention. In view 117, the balloon 113 is inflated by providing fluid through the inflation lumen and out through the inflation inlet 110 (not visible in view 117). The balloon locating features 111 are configured to hold the balloon even when inflated. To deflate the balloon, the fluid may be aspirated out of the inflation lumen. Post-procedure, the potentially cheaply-produced balloon 113 may be discarded, while the more expensive endoscopic device 101 may be sterilized and re-used. After a predetermined number of uses, the endoscopic device 101 may be disposed as well.

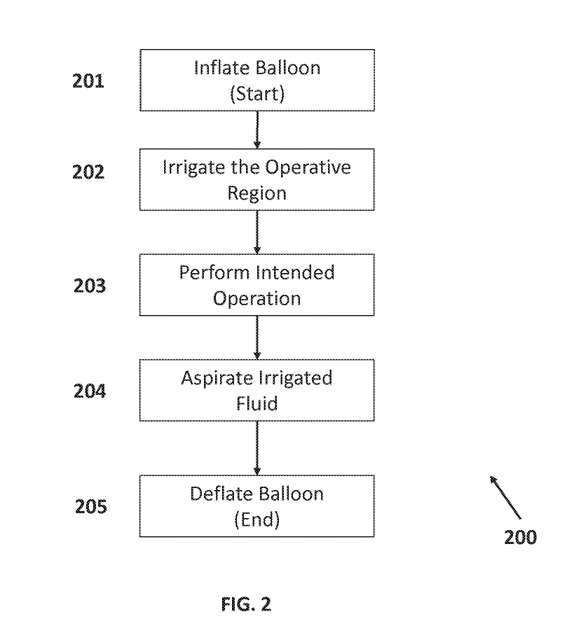

FIG. 2 illustrates a protocol for using the endoscopic device with a balloon attachment, such as endoscopic device 101 and balloon 113 from FIG. 1A-1E, within an anatomical lumen, to prevent unwanted fluid in non-operative regions in accordance with an embodiment of the invention. As shown in process 200, there are a series of steps to appropriately use a balloon attachment within a patient's body. However, prior to engaging in process 200, the distal end of the endoscopic device must first be positioned near the operative site in the correct anatomical lumen. Localization of the distal end 104 of the device 101 may be achieved using visual cues through the distally-mounted camera 106, fluoroscopy, electromagnetic imaging, or any other of a number of techniques.

Having located the distal end 104 of the endoscopic device 101 within the desired anatomical lumen (e.g., bronchiole), and having positioned the distal end 104 near the operative site, in step 201, the balloon 113 may be inflated by conveying fluid down the inflation lumen and through the inflation inlet 104. The balloon 113 may be inflated to the extent necessary to isolate the operative site and block the bronchiole. Confirmation of proper inflation may be confirmed using a variety of techniques, including fluoroscopy. Using fluoroscopy, the inflation fluid used in the balloon may be a combination of saline and contrast (e.g., 50/50 mixture) to enhance visibility.

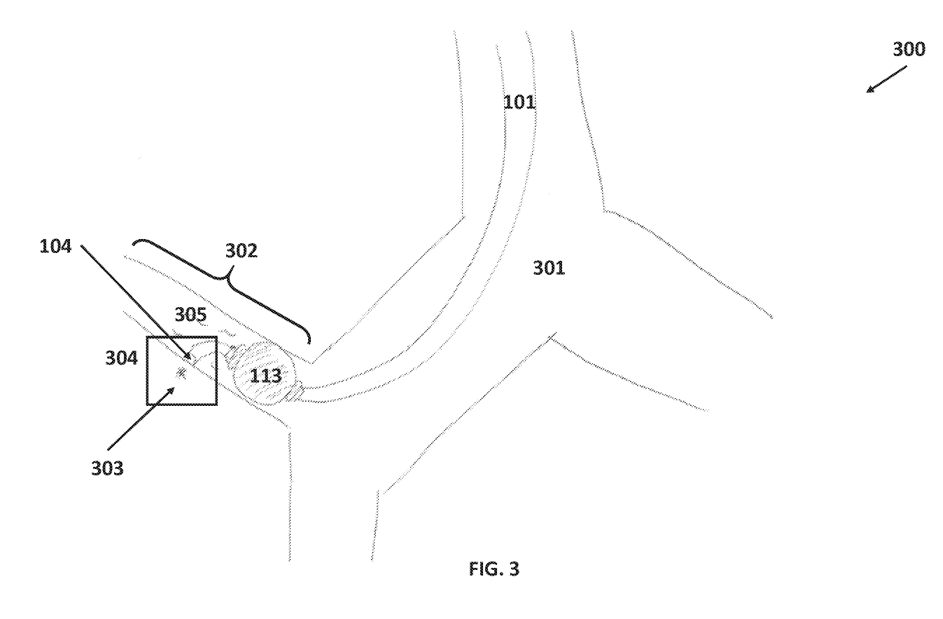

FIG. 3 illustrates use of an endoscopic device with a balloon attachment within an anatomical lumen. Specifically, internal view 300 from FIG. 3 illustrates how endoscopic tool 101 may be used within an anatomical lumen, such as a secondary bronchiole 302 off from the central airways 301. As shown in view 300, endoscopic device 101 may be positioned such that the distal tip 104 may be near the operative region 304, close to a lesion 303. As shown in view 300, inflating balloon 113 isolates the operative region 304 by blocking bronchiole 302.

Having properly inflated the balloon 113, and thus isolated the operative site 304 by blocking the anatomical lumen (302), in step 202, the operative region may be safely irrigated using irrigation ports 108 to improve the efficacy and efficiency of the subsequent clinical operation. As shown in view 300 from FIG. 3, when properly inflated, balloon 113 should prevent irrigated fluid 305 from endoscopic tool 101 from unintentionally entering the central airways 301. With the operative region 304 flooded, endoscopic tool 101 may then perform the desired procedure in step 203 from FIG. 2.

Steps 204 and 205 from FIG. 2 generally represent clean up after the procedure is completed in step 203. With the procedure complete, the irrigated fluid (305 in FIG. 3) may be aspirated by endoscopic tool 101 using any number of distally-mounted ports, such as working channel 109. After aspirating the fluid, the balloon 113 may be deflated by aspirating the fluid back through the inflation inlet 110 or by relieving fluid pressure in the inflation lumen. Having aspirated the irrigated fluid 305 and deflated the balloon 113, endoscopic device 101 and its distal tip 104 may be repositioned for either the next operative site or evacuated from the patient's body.

Beyond bronchoscopy, the present invention may be applied to a variety of other procedures, such as gastrointestinal and urology. For example, in gastrointestinal procedures, the anatomical lumens are much larger-using a balloon may assist stabilizing the flexible endoscopic device near the operative area.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Elements or components shown with any embodiment herein are exemplary for the specific embodiment and may be used on or in combination with other embodiments disclosed herein. While the invention is susceptible to various modifications and alternative forms, specific examples thereof have been shown in the drawings and are herein described in detail. The invention is not limited, however, to the particular forms or methods disclosed, but to the contrary, covers all modifications, equivalents and alternatives thereof.

What is claimed is:

1. An endoscopic apparatus comprising:
an elongate shaft comprising a steerable distal tip;
an expandable element coupled to the elongate shaft proximal of the steerable distal tip; and
an instrument base coupled to a proximal end of the elongate shaft and configured to be coupled to a robotic system so that the robotic system is able to articulate the steerable distal tip through the instrument base.

2. The endoscopic apparatus of claim 1, further comprising one or more pull wires enclosed at least partially within the elongate shaft, the one or more pull wires being configured to articulate the steerable distal tip.

3. The endoscopic apparatus of claim 1, wherein the elongate shaft comprises one or more of an inflation channel or lumen for expanding the expandable element, an irrigation channel, an aspiration channel, or a working channel.

4. The endoscopic apparatus of claim 1, wherein the steerable distal tip comprises an imaging source.

5. The endoscopic apparatus of claim 4, wherein the imaging source comprises one or more of a camera or a light emitter.

6. The endoscopic apparatus of claim 1, wherein the expandable element is removably coupled to the elongate shaft.

7. The endoscopic apparatus of claim 6, wherein the elongate shaft comprises one or more locating features to facilitate positioning the expandable element at a desired location on the elongate shaft proximal of the steerable distal tip.

8. The endoscopic apparatus of claim 6, wherein the expandable element is slidable over the elongate shaft.

9. The endoscopic apparatus of claim 6, wherein the expandable element comprises an inflatable balloon.

10. The endoscopic apparatus of claim 9, wherein the elongate shaft has one or more inflation inlets open to an interior of the inflatable balloon proximal of the steerable distal tip.

11. A method of performing endoscopic surgery, the method comprising:
providing an endoscopic apparatus;
advancing the endoscopic apparatus through an anatomical lumen;
expanding an expandable element on the endoscopic apparatus in the anatomical lumen; and
articulating a steerable distal tip of the endoscopic apparatus to perform a procedure on an operative site in the anatomical lumen,
wherein the steerable distal tip is articulated using a robotic system operatively coupled to an instrument base of the endoscopic apparatus.

12. The method of claim 11, wherein providing the endoscopic apparatus comprises coupling the expandable element to an elongate shaft of the endoscopic apparatus.

13. The method of claim 12, wherein coupling the expandable element to the elongate shaft comprises sliding the expandable element over the elongate shaft so the expandable element couples to one or more locating features of the elongate shaft.

14. The method of claim 11, wherein the robotic system advances the endoscopic apparatus through the anatomical lumen.

15. The method of claim 11, wherein expanding the expandable element in the anatomical lumen comprises stabilizing the endoscopic apparatus in the anatomical lumen.

16. The method of claim 11, wherein expanding the expandable element in the anatomical lumen comprises isolating the operative site in the anatomical lumen.

17. The method of claim 11, wherein expanding the expandable element in the anatomical lumen comprises inflating the expandable element.

18. The method of claim 11, further comprising irrigating the operative site with the endoscopic apparatus.

19. The method of claim 18, wherein the operative site is irrigated after the operative site has been isolated.

20. The method of claim 11, further comprising aspirating the operative site.

21. The method of claim 20, wherein the operative site is aspirated after the procedure has been performed on the operative site.

22. The method of claim 11, further comprising contracting the expandable element after the procedure has been performed on the operative site.

23. The method of claim 22, further comprising retracting the endoscopic apparatus from the anatomical lumen after the expandable element has been contracted.

24. The method of claim 11, wherein the anatomical lumen comprises one or more of a blood vessel, a bronchus, a bronchiole, a colon, an ear canal, an esophagus, a mouth, a nasal cavity, a rectum, a trachea, a ureter, a urethra, a uterus, or a vagina.

25. The method of claim 11, further comprising visualizing the anatomical lumen with the endoscopic apparatus.

26. The method of claim 11, further comprising de-coupling the expandable element from the endoscopic apparatus after the procedure has been performed on the operative site.

27. The method of claim 26, further comprising discarding the expandable element after the expandable element has been de-coupled from the endoscopic apparatus.

28. The method of claim 27, further comprising sterilizing and re-using the endoscopic apparatus after the expandable element has been discarded.

29. An endoscopic apparatus comprising:
an elongate shaft comprising a steerable distal tip, one or more locating features proximal of the steerable distal tip, one or more inflation inlets adjacent the one or more locating features, and a working channel;
an inflatable element configured to be slid over the elongate shaft to removably couple to the elongate shaft at the one or more locating features, thereby placing the inflatable element in fluid communication with the one or more inflation inlets;
an instrument base coupled to a proximal end of the elongate shaft and configured to be coupled to a robotic system so that the robotic system is able to articulate the steerable distal tip through the instrument base; and
an imaging source at the steerable distal tip.

30. A method of performing endoscopic surgery, the method comprising:
assembling an endoscopic apparatus by removably coupling a first inflatable element to one or more locating features on an elongate shaft, the coupled first inflatable element being in fluid communication with one or more inflation inlets on the elongate shaft;
coupling an instrument base of the endoscopic apparatus to a robotic system, wherein the instrument base is coupled to a proximal end of the elongate shaft, and wherein robotic system is operable with the endoscopic apparatus to one or more of advance the endoscopic apparatus into an anatomical lumen, retract the endoscopic apparatus from the anatomical lumen, visualize the anatomical lumen, inflate the first inflatable element, deflate the first inflatable element, perform a procedure with a steerable distal tip, irrigate the anatomical lumen, or aspirate the anatomical lumen;
de-coupling the first inflatable element from the endoscopic apparatus; and
discarding the first inflatable element while re-using the endoscopic apparatus with a second inflatable element.
